(19) United States
(12) Patent Application Publication
(10) Pub. No.: US 2006/0009480 A1
(43) Pub. Date: Jan. 12, 2006

(54) Sinomenine and sinomenine compounds, synthesis and use

(76) Inventors: Guo-Wei Qin, Shanghai (CN); Xi-Can Tang, Shanghai (CN); Rui Wang, Shanghai (CN); Tian-Xi Zhou, Shanghai (CN); Pierre Lestage, La Celle-Saint-Cloud (FR); Daniel-Henri Caignard, Le Pecq (FR); Pierre Renard, Le Chesnay (FR)

Correspondence Address: Hueschen and Sage;Attorneys and Counselors, 500 Columbia Plaza, 350 East MIchigan Avenue, Kalamazoo, MI, 49007-3856, US

(21) Appl. No.: 10/536,613

(22) PCT Filed: Nov. 26, 2003
(86) PCT No.: PCT/EP03/14841

§ 371 Date: May 25, 2005

(30) Foreign Application Priority Data
Nov. 28, 2002 (CN) 021538190

(51) Int. Cl. C07D 221/28; A61K 31/485

(52) U.S. Cl. 514/286; 546/74

(57) ABSTRACT

The invention relates to sinomenine and compounds thereof and also to compounds of formula (I): 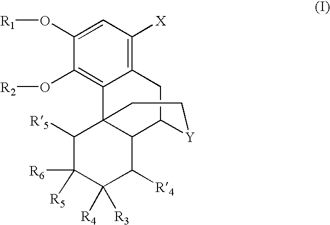 wherein
R₁ represents alkyl,
R₂ represents hydrogen or alkylcarbonyl, haloalkylcarbonyl or arylcarbonyl,
   Y represents a group 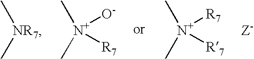
R₃, R₄, R′₄, R₅, R′₅ and R₆ are as defined in the description, and X represents halogen and medicinal products containing the same which are useful in treating mnemocognitive disorders.

### FIELD OF THE INVENTION

The invention relates to sinomenine and sinomenine derivatives and their use in mnemocognitive disorders.

### BACKGROUND AND PRIOR ART OF THE INVENTION

Sinomenum acutum is a plant taking the form of a ligneous liana, which is widespread in the centre, South-East and South-West of China and is included in the Chinese Pharmacopoeia (Pharmacopoeia Committee of People's Republic of China, 2000). It contains a large number of alkaloids of various chemical structures, such as sinomenine, sinoacutine, ethylsinomenine, disinomenine, tetrahydroepiberberine, tuduranine and magnoflorine (Huang Tai-Kang, <<Handbook of the Composition and Pharmacology of Common Chinese Drugs>>, Chinese Medical Science and Technology Publisher, 1994, Beijing, 1156-1160).

Sinomenine, a morphine-like alkaloid and a major constituent of the plant, has been much studied; in particular, it has been possible to demonstrate anti-inflammatory, immunosuppressive, anti-arrhythmic and analgesic properties (Qiang Liu et al., Chinese Traditional and Herbal Drugs, 1997, 28(4), 247).

We have now discovered that sinomenine has mnemocognition-facilitating properties in animal experimental models.

Ageing of the population due to increased life expectancy has brought with it a major increase in cognitive disorders associated with normal cerebral ageing or pathological cerebral ageing occurring in the course of neurodegenerative diseases such as, for example, Alzheimer's disease.

The majority of substances used today in treating cognitive disorders associated with ageing act by facilitating the central cholinergic systems—either directly, as in the case of acetylcholinesterase inhibitors (tacrine, donepezil) and cholinergic agonists (nefiracetam), or indirectly, as in the case of nootropic agents (piracetam, pramiracetam) and cerebral vasodilators (vinpocetine).

It has been therefore been especially valuable to synthesise new compounds that are capable of opposing the cognitive disorders associated with ageing and/or of improving cognitive processes.

The present invention relates, on the one hand, to the use of sinomenine: 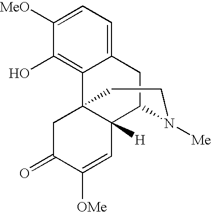
and/or sinomenine compounds in mnemocognitive disorders and, on the other hand, to the synthesis of new compounds having especially valuable pharmacological properties in the same area.

### DETAILED DESCRIPTION OF THE INVENTION

The present invention relates more specifically to compounds of formula (I): 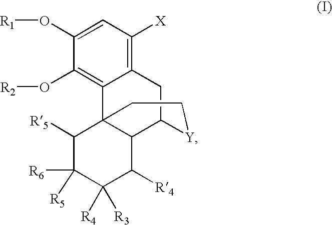
wherein
R₁ represents an alkyl group,
R₂ represents a hydrogen atom or an alkylcarbonyl group, an haloalkylcarbonyl group or an arylcarbonyl group,
Y represents a group 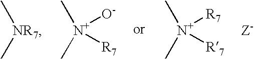
   wherein R₇ and R′₇, identical or differents, each represent an alkyl group, and Z⁻ represents a halogen anion,
R₃ represents a hydroxy or alkoxy group,
R₄ and R′₄ each represent a hydrogen atom or together form an additional bond, or R₃ and R₄ together form an oxo or ═N—OR₈ group (wherein R₈ represents a hydrogen atom or an alkyl group),
R₆ represents a hydroxy, alkylcarbonyloxy (wherein the alkyl moiety can be substituted by a hydroxy, alkoxy, carboxy or alkyloxycarbonyl group) or alkoxy group,
R₅ and R′₅ each represent a hydrogen atom or together form an additional bond, or R₅ and R₆ together form an oxo, ═N—OR₉ or ═N—NR₁₀R₁₁ group (wherein R₉, R₁₀, and R₁₁, which may be the same or different, each represent a hydrogen atom or an alkyl group),
and X represents a halogen atom,
   with the proviso that the compound of formula (I) cannot represent 1-bromo-4-hydroxy-3,7-dimethoxy-17-methyl-7,8-didehydromorphinan-6-one, it being understood that
“alkyl” means an alkyl group containing 1 to 6 carbon atoms which may be linear or branched,
“alkoxy” means an alkyloxy group containing 1 to 6 carbon atoms which may be linear or branched,
   to their enantiomers and diastereoisomers, and to addition salts thereof with a pharmaceutically acceptable acid or base.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, methanesulphonic acid, camphoric acid, oxalic acid etc.

Among the pharmaceutically acceptable bases there may be mentioned, without implying any limitation, sodium hydroxide, potassium hydroxide, triethylamine, tert-butylamine etc.

The preferred configuration of compounds of formula (I) according to the invention is that shown in formula (I′):

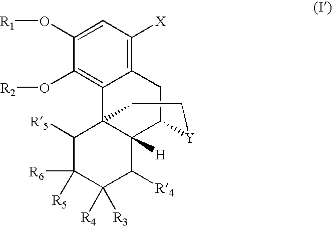

The preferred group R₁ is the methyl group.

Advantageously, R₂ represents a hydrogen atom or a group ETCO and more preferably a hydrogen atom.

Y represents, preferably, a group NR₇ 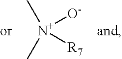
more expecially, a group

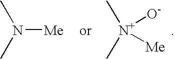

X represents, very preferably, a chlorine or bromine atom.

Advantageously, the invention relates to compounds of formula (I) wherein R₃ represents an alkoxy group and R₄ and R′₄ together form an additional bond.

The preferred meaning of R₅ is a hydrogen atom.

R₆ represents advantageously an OH, ethoxy or alkylcarbonyloxy group and, more especially, ethylcarbonyloxy.

Another interesting aspect of the invention is compounds of formula (I) for which R₅ and R₆ form together an oxo group or an ═N—OH group.

Very preferably, the invention relates to compounds of formula (I″) and (I′″) 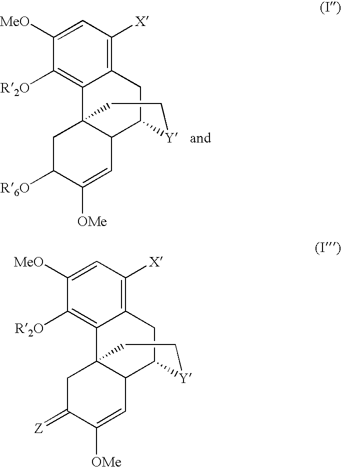
wherein Y′ represents 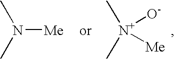
R′₂ and R′₆, which may be the same or different, represent a hydrogen atom or an alkylcarbonyl group, X′ represents a chlorine or bromine atom and Z represents ═O or ═N—OH.

Even more preferably, the invention relates to compounds of formula (I) that are (9α,13α)-1-chloro-3,7-dimethoxy-17-methyl-7,8-didehydromorphinan-4,6-diol, (9α,13α)-1-chloro-3,7-dimethoxy-17-methyl-4-(propionyloxy)-7,8-didehydromorphinan-6-yl propionate, (9α,13α)-1-bromo-3,7-dimethoxy-17-methyl-7,8-didehydromorphinan-4,6-diol, (9α,13α)-1-bromo-4-hydroxy-3,7-dimethoxy-17-methyl-7,8-didehydromorphinan-6-one oxime, (9α,13α)-1-bromo-4-hydroxy-3,7-dimethoxy-17-methyl-7,8-didehydromorphinan-6-one N-oxide, (9α,13α)-1-chloro-4-hydroxy-3,7-dimethoxy-17-methyl-7,8-didehydromorphinan-6-one N-oxide.

The enantiomers and diastereoisomers and addition salts with a pharmaceutically acceptable acid or base of the preferred compounds of the invention form an integral part of the invention.

The invention relates also to a process for the preparation of compounds of formula (I), which process is characterised in that there is used as starting material the compound of formula (II) 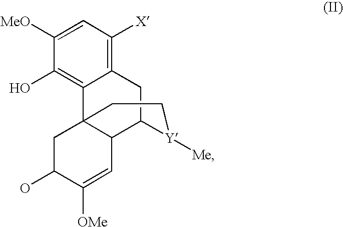
obtained by extraction starting from the stem of Sinomenum acutum according to FIG. 1 below: [image]
which is subjected to the action of a halogenating agent such as SO₂Cl₂ or Br₂ to obtain the compound of formula (I/a), a particular case of the compounds of formula (I): 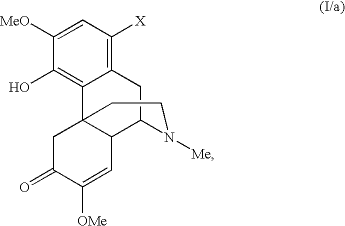
wherein X is as defined for formula (I), which compound of formula (I/a) is subjected to conventional chemical reactions to obtain the totality of the compounds of formula (I), which may be purified according to a conventional separation technique, are converted, if desired, into their addition salts with a pharmaceutically acceptable acid or base and are separated, where appropriate, into their isomers according to a conventional separation technique.

Besides the fact that the compounds of the present invention are new, they possess properties of facilitating cognitive processes, making them of use in the treatment of cognitive deficiencies associated with cerebral ageing and with neurodegenerative diseases such as Alzheimer's disease, Parkinson's disease, Pick's disease, Korsakoff's disease, and frontal lobe and subcortical dementias.

The invention relates also to pharmaceutical compositions comprising as active ingredient at least one compound of formula (I) together with one or more appropriate, inert, non-toxic excipients.

The Applicant has moreover discovered that sinomenine and/or sinomenine compounds have mnemocognition-facilitating properties.

The invention accordingly relates also to the use of sinomenine and/or sinomenine compounds in obtaining pharmaceutical compositions for use in the treatment of cognitive deficiencies associated with cerebral ageing and with neurodegenerative diseases such as Alzheimer's disease, Parkinson's disease, Pick's disease, Korsakoff's disease, and frontal lobe and subcortical dementias.

More especially, the invention relates to the use, in obtaining pharmaceutical compositions for use in the treatment of cognitive deficiencies associated with cerebral ageing and with neurodegenerative diseases, of sinomenine and/or sinomenine compounds such as, for example, the compounds of formula (Ia): 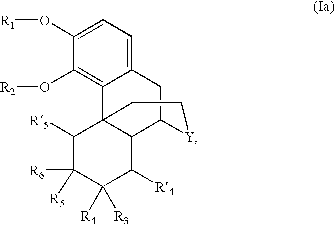
wherein R₁, R₂, R₃, R₄, R′₄, R₅, R′₅, R₆ and Y are as defined for formula (I), and, more especially, of (9α,13α)-4-hydroxy-3,7-dimethoxy-17-methyl-7,8-didehydromorphinan-6-one hydrazone; (7α,9α,13α)-4-hydroxy-3,7-dimethoxy-17-methylmorphinan-6-one; (7β,9α,13α)-4-hydroxy-3,7-dimethoxy-17-methylmorphinan-6-one; (9α,13α)-3,7-dimethoxy-17-methyl-6-oxo-7,8-didehydromorphinan-4-yl propionate; (9α,13α)-3,4,7-tri-methoxy-17-methyl-7,8-didehydromorphinan-6-one; (9α,13α)-4-hydroxy-3,7-dimethoxy-17-methyl-7,8-didehydromorphinan-6-one oxime; (9α,13α)-3,7-dimethoxy-17-methyl-7,8-didehydromorphinan-4,6-diol; (9α,13α)-4-hydroxy-3,7-dimethoxy-17-methyl-7,8-dide-hydromorphinan-6-one N-oxide; (9α,13α)-6-amino-3,7-dimethoxy-17-methylmorphinan-4-ol; 4-{[(9α,13α)-4-hydroxy-3,7-dimethoxy-17-methyl-7,8-didehydromorphinan-6-yl]-oxy}-4-oxobutanoic acid; (9α,13α)-3,7-dimethoxy-17-methyl-4-(propionyloxy)-7,8-dide-hydromorphinan-6-yl propionate; (9α,13α)-17-benzyl-4-hydroxy-3,7-dimethoxy-17-methyl-7,8-didehydromorphinan-17-ium-6-one bromide; (9α,13α)-17-ethyl-3,7-dimethoxy-17-methyl-7,8-didehydromorphinan-17-ium-4,6-diol bromide; (9α,13α)-17-3,7-ethyl-4-hydroxy-3,7-dimethoxy-17-methyl-7,8-didehydromorphinan-17-ium-6-one bromide; (9α,13α)-4-(benzoyloxy)-3,7-dimethoxy-17-methyl-7,8-didehydromorphinan-6-yl benzoate; (9α,13α)-4-hydroxy-3,7-dimethoxy-17-methyl-7,8-didehydromorphinan-6-yl benzoate; (9α,13α)-6-hydroxy-3,7-dimethoxy-17-methyl-7,8-didehydromorphinan-4-yl benzoate.

An advantageous aspect of the invention relates to the use of sinomenine in obtaining pharmaceutical compositions for use in the treatment of cognitive deficiencies associated with cerebral ageing and with neurodegenerative diseases.

Another especially interesting aspect of the invention relates to the use, in obtaining pharmaceutical compositions for use in the treatment of cognitive deficiencies associated with cerebral ageing and with neurodegenerative diseases, of compounds of formula (Ia) and, more especially, of (9α,13α)-4-hydroxy-3,7-dimethoxy-17-methyl-7,8-didehydromorphinan-6-one hydrazone; of (7α,9α,13α)-4-hydroxy-3,7-dimethoxy-17-methylmorphinan-6-one; of (7β,9α,13α)-4-hydroxy-3,7-dimethoxy-17-methylmorphinan-6-one; of (9α,13α)-3,7-dimethoxy-17-methyl-6-oxo-7,8-didehydromorphinan-4-yl propionate; of (9α,13α)-3,4,7-trimethoxy-17-methyl-7,8-didehydromorphinan-6-one; of (9α,13α)-4-hydroxy-3,7-dimethoxy-17-methyl-7,8-didehydromorphinan-6-one oxime; of (9α,13α)-3,7-dimethoxy-17-methyl-7,8-didehydromorphinan-4,6-diol; of (9α,13α)-4-hydroxy-3,7-dimethoxy-17-methyl-7,8-didehydromorphinan-6-one N-oxide; of (9α,13α)-6-amino-3,7-dimethoxy-17-methylmorphinan-4-ol; of 4-{[(9α,13α)-4-hydroxy-3,7-dimethoxy-17-methyl-7,8-didehydromorphinan-6-yl]oxy}-4-oxobutanoic acid; of (9α,13α)-3,7-dimethoxy-17-methyl-4-(propionyloxy)-7,8-didehydromorphinan-6-yl propionate; of (9α,13α)-17-benzyl-4-hydroxy-3,7-dimethoxy-17-methyl-7,8-didehydromorphinan-17-ium-6-one bromide; of (9α,13α)-17-ethyl-3,7-dimethoxy-17-methyl-7,8-didehydromorphinan-17-ium-4,6-diol bromide; of (9α,13α)-17-ethyl-4-hydroxy-3,7-dimethoxy-17-methyl-7,8-didehydromorphinan-17-ium-6-one bromide; of (9α,13α)-4-(benzoyloxy)-3,7-dimethoxy-17-methyl-7,8-didehydromorphinan-6-yl benzoate; of (9α,13α)-4-hydroxy-3,7-dimethoxy-17-methyl-7,8-didehydromorphinan-6-yl benzoate; of (9α,13α)-6-hydroxy-3,7-dimethoxy-17-methyl-7,8-didehydromorphinan-4-yl benzoate.

The invention relates also to pharmaceutical compositions comprising sinomenine or a compound thereof, in combination with one or more pharmaceutically acceptable excipients, for use in the treatment of cognitive deficiencies associated with cerebral ageing and with neurodegenerative diseases such as Alzheimer's disease, Parkinson's disease, Pick's disease, Korsakoff's disease, and frontal lobe and subcortical dementias.

Among the pharmaceutical compositions according to the invention, there may be mentioned more especially those that are suitable for oral, parenteral (intravenous or subcutaneous) or nasal administration, tablets or dragees, sublingual tablets, gelatin capsules, lozenges, suppositories, creams, ointments, dermal gels, injectable preparations, drinkable suspensions etc.

The useful dosage can be varied according to the nature and severity of the disorder, the administration route and also the age and weight of the patient. The dosage varies from 0.01 mg to 1 g per day in one or more administrations.

The following Examples illustrate the invention but do not limit it in any way.

### EXAMPLE 1

### (9α,13α)-1-Chloro-4-hydroxy-3,7-dimethoxy-17-methyl-7,8-didehydromorphinan-6-one

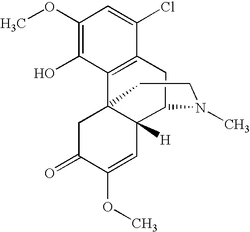

To a solution of 100 mg of the compound of formula (II) in 5 ml of CHCl₃ there are added 3 drops of SO₂Cl₂. The reaction mixture is stirred at ambient temperature for 4 hours and the pH is adjusted to 7-8 with NaHCO₃ solution; extraction with CHCl₃ is then carried out. The organic phase is evaporated under reduced pressure and the residue obtained is chromatographed on silica gel using an eluant CHCl₃-MeOH (9:1) to yield the title compound in the form of a yellowish solid.

Melting point: 126-128° C.

### EXAMPLE 2

### (9α,13α)1-Chloro-3,7-dimethoxy-17-methyl-6oxo-7,8-didehydromorphinan-4-yl propionate

To a solution of 500 mg of the compound obtained in Example 1 and 100 mg of DMAP in 15 ml of pyridine there are slowly added 2 ml of propionic anhydride, and the reaction mixture is stirred at ambient temperature for 3 hours. The reaction mixture is then evaporated and the residue obtained is dissolved in a small volume of water. The solution obtained is adjusted to pH=8-9 with NaHCO₃ solution and is then extracted with CHCl₃. The organic phase is washed 3 times with water, dried over sodium sulphate and evaporated. The residue obtained is chromatographed on silica gel using an eluant CHCl₃-MeOH (20:1) to yield the title compound in the form of a colourless oil.

### EXAMPLE 3

### (60β,7β,9α,13α)1-Chloro-3,7-dimethoxy-17-methylmorphinan4,6-diol

A mixture of 720 mg of compound of Example 1 and 100 mg of PtO₂ in 50 ml of absolute ethanol is stirred at room temperature under H₂ atmosphere for 12 hours. The PtO₂ is removed by filtration and the ethanol is evaporated in vacuum to give a syrupy residue. This residue is washed with hot absolute ethanol (10 ml) to give a powdery solid which is collected by filtration and crystallized in CHCl₃/C₂H₅OH to give the title compound in the form of white crystals.

Melting point: 210-212° C.

### EXAMPLE 4

### (9α,13α)-1-Chloro-3,7-dimethoxy-17-methyl-7,8-didehydromorphinan-4,6-diol

To a solution of 500 mg of the compound obtained in Example 1 in 15 ml of methanol there are added 500 mg of NaBH₄, and the reaction mixture is stirred for 1.5 hours. The methanol is then evaporated off and the residue obtained is extracted with CHCl₃. The organic phase is dried over Na₂SO₄ and evaporated under reduced pressure. The title compound is obtained in the form of white crystals, by recrystallisation from Et₂O.

Melting point: 118-120° C.

### EXAMPLE 5

### (9α,13α)-1-Chloro-3,7-dimethoxy-17-methyl4-(propionyloxy)7,8-didehydromorphinan-6-yl propionate

The title compound is obtained using the procedure described for Example 2, starting from the compound obtained in Example 4.

Oil.

### EXAMPLE 6

### (6β,7β,9α,13α)-1-Chloro-3,7-dimethoxy-17-methyl-4-(propionyloxy)-morphinan-6-yl propionate

The title compound is obtained using the procedure described for Example 2, starting from the compound obtained in Example 3.

Oil.

### EXAMPLE 7

### (9α,13α)-1-Chloro-3,4,7-trimethoxy-17-methyl-7,8-didehydromorphinan-6-one

A solution of 400 mg of the compound of Example 1 in 10 ml of methanol is treated with an excess of a freshly made preparation of diazomethane in ether, and the reaction mixture is stirred at ambient temperature for 12 hours. The excess of diazomethane is then broken down using glacial acetic acid, and the solvents are evaporated off under reduced pressure. The residue obtained is adjusted to pH=8-9 using saturated NaHCO₃ solution and is then extracted with CHCl₃. The organic phase is dried over Na₂SO₄ and evaporated in vacuo, and the residue obtained is chromatographed on silica gel (eluant CHCl₃-MeOH) to yield the title product in the form of an oil.

### EXAMPLE 8

### (9α,13α)-1-Chloro-3,4,7-trimethoxy-17-methyl-7,8-didehydromorphinan-6-ol

The title compound is obtained using the procedure described for Example 4, starting from the compound obtained in Example 7. Colourless crystals. Melting point: 163-165° C.

### EXAMPLE 9

### (9α,13α)-1-Chloro-4-hydroxy-3,7-dimethoxy-17-methyl-7,8-didehydromorphinan-6-one oxime

To a solution of 360 mg of the compound obtained in Example 1 in ethanol there are added 200 mg of NH₂OH. HCl and 300 mg of sodium acetate. The reaction mixture is stirred for 4 hours; it is then filtered and evaporated under reduced pressure. The residue obtained is made alkaline using NaHCO₃ solution and extracted with CHCl₃. The organic phase is dried over Na₂SO₄ and then evaporated under reduced pressure, and the title compound is obtained in the form of needles, by recrystallisation from EtOH.

Melting point: 167-169° C.

### EXAMPLE 10

### (9α,13α)-1-Chloro6-ethoxy-4-hydroxy-3-methoxy-17-methyl-5,6-didehydromorphinan-7-one

To a solution of 1.3 g of the compound obtained in Example 1 in 100 ml of CHCl₃ and 10 ml of absolute alcohol there is added SO₂Cl₂ at 10° C., and the reaction mixture is stirred for 8 hours. The solvent is then evaporated off under reduced pressure, and the residue is neutralised using NaHCO₃ and then extracted with CHCl₃. The organic phase is dried over Na₂SO₄ and evaporated under reduced pressure, and the title compound is obtained in the form of yellowish crystals, by recrystallisation from CH₃CN.

Melting point: 190-192° C.

### EXAMPLE 11

### (9α,13α)-1-Chloro4-hydroxy-3,7-dimethoxy-17-methyl-7,8-didehydromorphinan-6-one hydrazone

A solution of 600 mg of the compound obtained in Example 1 in 10 ml of 85% hydrazine is stirred at 90° C. for 8 hours. After cooling, the reaction mixture is filtered and the solid obtained is washed with water and recrystallised from EtOH to yield the title compound in the form of yellowish crystals.

Melting point: 235-237° C.

### EXAMPLE 12

### (9α,13α)-1-Bromo-4-hydroxy-3,7-dimethoxy-17-methyl-7,8-didehydromorphinan-6-one

A solution of 6.6 g of the compound obtained in Example 1 in 150 ml of CHCl₃ is cooled to 0° C., and bromine dried over concentrated sulphuric acid is added dropwise, with stirring, whilst maintaining the reaction mixture at 5° C. The reaction is continued for a few minutes and then neutralisation is carried out using NaHCO₃. The organic phase is separated off, dried over Na₂SO₄ and evaporated under reduced pressure, and the residue obtained is recrystallised from EtOH to yield the title compound in the form of brown crystals.

Melting point: 163-165° C.

### EXAMPLE 13

### (9α,13α)-1-Bromo-3,7-dimethoxy-17-methyl-7,8-didehydromorphinan-4,6-diol

The title compound is obtained using the procedure described for Example 4, starting from the compound obtained in Example 12 and replacing NaBH₄ by KBH₄. Solid.

Melting point: 144-146° C.

### EXAMPLE 14

### (9α,13α)-1-Bromo-4-hydroxy-3,7-dimethoxy-17-methyl-7,8-didehydromorphinan-6-one hydrazone

The title compound is obtained using the procedure described for Example 11, starting from the compound obtained in Example 12. Solid.

Melting point: 208-210° C.

### EXAMPLE 15

### (9α,13α)-1-Bromo4-hydroxy-3,7-dimethoxy-17-methyl-7,8-didehydromorphinan-6-one oxime

The title compound is obtained using the procedure described for Example 9, starting from the compound obtained in Example 12. Solid.

Melting point: 180-182° C.

### EXAMPLE 16

### (9α,13α)1-Bromo-4-hydroxy-3,7-dimethoxy-17-methyl-7,8-didehydromorphinan-6-one N-oxide

A mixture of compound of Example 12 (820 mg) in H₂O₂ (10 ml) is stirred at room temperature for 24 hours and then extracted with CHCl₃ three times (30 ml×3). The combined extracts are dried overnight with anhydrous Na₂SO₄ and the solvent is removed by evaporation to give a residue to which are added 30 ml of cold water. The powdery solid is collected by filtration, washed with cold water until the water being colorless, and crystallized in ethanol to give the title compound as a solid.

Melting point: 170-172° C.

### EXAMPLE 17

### (9α,13α)-1-Chloro-4-hydroxy-3,7-dimethoxy-17-methyl-7,8-didehydromorphinan-6-one N-oxide

A mixture of compound of Example 1 (720 mg) in H₂O₂ (10 ml) is stirred at room temperature for 24 hours and then extracted three times (25 ml×3). The combined extracts are dried over anhydrous Na₂SO₄ and the solvent is removed by evaporation. 30 ml of cold water are added to the residue obtained and the resulting powdery white solid is collected by filtration and crystallized in ethanol to give the title compound as a solid.

Melting point: 170-172° C.

### EXAMPLE 18

### (9α,13α)1-Bromo-3,7-dimethoxy-17-methylmorphinan4,6-diol

The title compound is obtained using the procedure described for Example 3, starting from the compound obtained in Example 12.

Melting point: 160-162° C.

### EXAMPLE 19

### (9α,13α)-1-Chloro-6-ethoxy-3-methoxy-17-methyl-5,6-didehydromorphinan-4,7-diol

The title compound is obtained using the procedure described for Example 4, starting from the compound obtained in Example 10 and replacing NaBH₄ by KBH₄. Solid.

Melting point: 168-170° C.

### EXAMPLE 20

### (9α,13α)-1-Chloro-6-ethoxy-4-hydroxy-3-methoxy-17-methyl-5,6-didehydromorphinan-7-one oxime

The title compound is obtained using the procedure described for Example 9, starting from the compound obtained in Example 10. Solid.

Melting point: 216-218° C.

### EXAMPLE 21

### (9α,13α)-17-Benzyl-1-bromo-4-hydroxy-3,7-dimethoxy-17-methyl-7,8-didehydromorphinan-17-ium-6-one bromide

The title compound is obtained from compound of example 12 subjected to the action of benzylbromide.

Melting point: 190-192° C.

### EXAMPLE 22

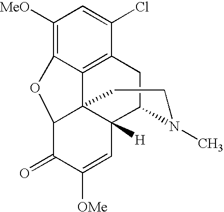

The title compound is obtained after treatment of compound of Example 1 in basic medium.

Melting point: 204-206° C.

### EXAMPLE 23

### (9α,13α)-1-Chloro-4-hydroxy-6-isopropoxy-3-methoxy-17-methyl-5,6-didehydromorphinan-7-one

The title compound is obtained using the procedure described in Example 10 replacing absolute alcohol with isopropylic alcohol.

Melting point: 216-218° C.

### EXAMPLE 24

### (9α,13α)-1-Chloro-3,7-dimethoxy-17-methyl-6oxo-7,8-didehydromorphinan-4-yl chloroacetate

The title compound is obtained using the procedure described in Example 2 replacing propionic anhydrid with chloroacetic anhydrid.

Melting point: 223-225° C.

### PHARMACOLOGICAL STUDY OF COMPOUNDS OF THE INVENTION

### EXAMPLE A

### Acute Toxicity Study

Acute toxicity was evaluated after oral administration to groups each comprising 8 mice (26±2 grams). The animals were observed at regular intervals during the course of the first day, and daily for the two weeks following treatment. The LD₅₀ (dose that causes the death of 50% of the animals) was evaluated and demonstrated the low toxicity of the compounds of the invention.

### EXAMPLE B

### Morris Water Maze Test in the Mouse

The anti-amnesic effects of the compounds of the present invention have been evaluated using the Morris water maze test (Morris et al., Nature,1986, 319, 774-776) in the mouse and scopolamine as amnesic agent. Kumming strain mice (18-24 g, Shanghai Experimental Animal Centre) of either sex were used. Mice were placed on the water maze (80×50×20 cm) and trained to find the platform. Following the period of one day's habituation, each mouse received 3 daily training sessions for seven days. Mice were trained to a criterion of finding the platform within 20 seconds and with <2 errors of entering a dead-end. Once a mouse met the criterion, training was reduced to one daily session until all mice met the criterion. Trained mice were randomly assigned to sub-groups. Compounds under study were dissolved in distilled water and administered by the oral route 40 minutes before behavioural testing. Scopolamine (5 mg/kg, i.p.) was injected 30 minutes before the test. The number of errors and the time for reaching the platform were recorded. Data were expressed as means±s.e.m. Statistical analysis was performed using ANOVA followed by Duncan's multiple-range test.

Results demonstrate that compounds of the present invention were capable of counteracting in a dose-dependent manner (from 20 to 100 mg/kg) scopolamine-induced memory impairments in the Morris water maze test in the mouse, indicating that such compounds possess anti-amnesic properties. As example, compound of Example 4 gave the following results:

$$\{ \begin{matrix}Scopolamine \text{:} & 0 \\ Example 4 \text{:} & 0\end{matrix} ⇒ Latency to find the platform = 15 s \\ \{ \begin{matrix}Scopolamine \text{:} & 3 mg \text{/} kg \\ Example 4 \text{:} & 0\end{matrix} ⇒ Latency to find the platform = 55 s \\ \{ \begin{matrix}Scopolamine \text{:} & 3 mg \text{/} kg \\ Example 4 \text{:} & 20 mg \text{/} kg\end{matrix} ⇒ Latency to find the platform = 35 s \\ \{ \begin{matrix}Scopolamine \text{:} & 3 mg \text{/} kg \\ Example 4 \text{:} & 30 mg \text{/} kg\end{matrix} ⇒ Latency to find the platform = 25 s$$

### EXAMPLE C

### Social Recognition in the Wistar Rat

Initially described in 1982 by THOR and HOLLOWAY (J. Comp. Physiol., 1982, 96, 1000-1006), the social recognition test has subsequently been proposed by various authors (DANTZER et al., Psychopharmacology, 1987, 91, 363-368; PERIO et al., Psychopharmacology, 1989, 97, 262-268) for studying the mnemocognitive effects of new compounds. The test is based on the natural expression of the olfactory memory of the rat and its natural tendency to forget, and allows evaluation of memorisation, by recognition of a young congeneric animal, by an adult rat. A young rat (21 days), taken at random, is placed for 5 minutes in the cage housing an adult rat. With the aid of a video device, the experimenter observes the social recognition behaviour of the adult rat and measures its overall duration. The young rat is then removed from the adult rat's cage and is placed in its own cage until the second introduction. The adult rat is given the compound under test and, after 2 hours, is again brought into the presence (5 minutes) of the young rat. The social recognition behaviour is then observed again and its duration measured. The assessment criterion is the difference (T₂−T₁), expressed in seconds, between the “recognition” times of the 2 encounters.

The results obtained show a difference (T₂−T₁) ranging from (−10) s to (−36) s for doses ranging from 3 to 30 mg/kg, which shows that the compounds of the invention very greatly enhance memorisation. As example, compound of Example 13 at a dose of 20 mg/kg showed a difference (T₂−T₁) of −36 s, and compound of Example 5 a (T₂−T₁) of −31 s.

### EXAMPLE D

### Object Recognition in the Wistar Rat

The object recognition test in the Wistar rat was initially developed by ENNACEUR and DELACOUR (Behav. Brain Res., 1988, 31, 47-59). The test is based on the spontaneous exploratory activity of the animal and has the characteristics of episodic memory in humans. This memory test is sensitive to ageing (SCALI et al., Eur. J. Pharmacol., 1997, 325, 173-180) and to cholinergic dysfunctions (BARTOLINI et al., Pharm. Biochem. Behav. 1996, 53(2), 277-283) and is based on the differences in the exploration of 2 objects of fairly similar shape—one familiar, the other new. Prior to the test, the animals are habituated to the environment (an enclosure without an object). In the course of a first session, the rats are placed (3 minutes) in the enclosure, in which there are 2 identical objects. The duration of exploration is measured for each object. In the course of the second session (3 minutes), 24 hours later, 1 of the 2 objects is replaced by a new object. The duration of exploration is measured for each object. The assessment criterion is the difference, Delta, expressed in seconds, between the exploration times for the new object and for the familiar object in the course of the second session. The control animals, previously treated with the carrier by the IP route 30 minutes before each session, explore the familiar object and the new object in an identical manner, which indicates that the object introduced earlier has been forgotten. Animals treated with a compound that facilitates mnemocognition preferentially explore the new object, which indicates that the object introduced earlier has been remembered.

The results obtained show a difference, Delta, ranging from 5 to 11 s, for doses ranging from 0.3 to 10 mg/kg, which shows that the compounds of the invention greatly enhance memorisation. As example, compound of Example 4 showed a Delta of 10 s at a dose of 3 mg/kg.

### EXEMPLE E

### NANO₂ Induced Anoxia in Mice

The neuroprotective effects of the compounds of the present invention have been evaluated in mice. Kunming strain mice of either sex were supplied by Shangha\dot{i} Experimental Animal Center, Chinese Academy of Sciences (Grade clear, Certificate N°005). Mice weighing 22-28 g were kept in a 12 hours-light-dark cycle and given food and water ad libitum. Compounds under study were dissolved in a 5% polysorbate-80 solution and orally administered (50 mg/kg) 60 minutes prior to the administration of NaNO₂ at a dose of 225 mg/kg ip. Lethality was observed and the prolongation of survival was recorded. The results obtained indicate that the compounds of the present invention were able to increase the survival of mice after an ip administration of NaNO₂. These results demonstrate that the compounds of the present invention possess patent anti-anoxic and neuroprotective effects in the mouse. As example, compound of Example 5 shows a prolongation of survival of 31% at 70 mg/kg p.o.

### EXAMPLE F

### Pharmaceutical Composition

Formula for the preparation of 1000 tablets each containing 10 mg of active ingredient:

| | | |
|---|---|---|
| (9α,13α)-1-Bromo-3,7-dimethoxy-17-methyl- | 10 | g |
| 7,8-didehydromorphinan-4,6-diol | | |
| hydroxypropylcellulose | 2 | g |
| wheat starch | 10 | g |
| lactose | 100 | g |
| magnesium stearate | 3 | g |
| talc | 3 | g |

1-30. (canceled)

31. A compound selected from those of formula (I): 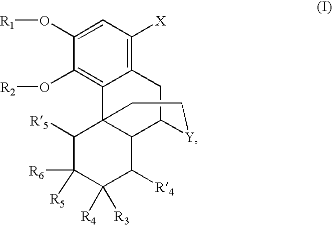
wherein
R₁ represents alkyl,
R₂ represents hydrogen, alkylcarbonyl, haloalkylcarbonyl or arylcarbonyl,
Y represents 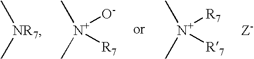
wherein R₇ and R′₇, which may be identical or different, each represent alkyl and Z⁻ represents a halogen anion,
R₃ represents hydroxy or alkoxy,
R₄ and R′₄ each represent hydrogen or together form an additional bond, or R₃ and R₄ together form oxo or ═N—OR₈ (wherein R₈ represents hydrogen or alkyl),
R₆ represents hydroxy, alkylcarbonyloxy (wherein the alkyl moiety may be substituted by hydroxy, alkoxy, carboxy or alkyloxycarbonyl) or alkoxy,
R₅ and R′₅ each represent hydrogen or together form an additional bond, or R₅ and R₆ together form oxo, ═N—OR₉ or ═N—NR₁₀R₁₁ (wherein R₉, R₁₀, and R₁₁, which may be the same or different, each represent hydrogen or alkyl),
and X represents halogen,
it being understood that:
the compound of formula (I) may not represent 1-bromo-4-hydroxy-3,7-dimethoxy-17-methyl-7,8-didehydromorphinan-6-one,
the term “alkyl” denotes an alkyl group having 1 to 6 carbon atoms which may be linear or branched, and
the term “alkoxy” denotes an alkyloxy group having 1 to 6 carbon atoms which may be linear or branched,
its enantiomers and diastereoisomers, and addition salts thereof with a pharmaceutically-acceptable acid or base.

32. A compound of claim 31, wherein R₁ represents methyl.

33. A compound of claim 31, wherein R₂ represents hydrogen.

34. A compound of claim 31, wherein R₂ represents alkylcarbonyl.

35. A compound of claim 31, wherein R₂ represents ethylcarbonyl.

36. A compound of claim 31, wherein Y represents NR₇.

37. A compound of claim 31, wherein Y represents

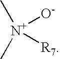

38. A compound of claim 31, wherein X represents chlorine.

39. A compound of claim 31, wherein X represents bromine.

40. A compound of claim 31, wherein R₃ represents alkoxy.

41. A compound of claim 31, wherein R₅ represents hydrogen.

42. A compound of claim 31, wherein R₆ represents OH.

43. A compound of claim 31, wherein R₆ represents alkylcarbonyloxy.

44. A compound of claim 31, wherein R₅ and R₆ together form oxo.

45. A compound of claim 31, wherein R₅ and R₆ together form ═N—OH.

46. A compound of claim 31, which is selected from (9α,13α)-1-chloro-3,7-dimethoxy-17-methyl-7,8-didehydromorphinan-4,6-diol and addition salts thereof with a pharmaceutically-acceptable acid or base.

47. A compound of claim 31, which is selected from (9α,13α)-1-chloro-3,7-dimethoxy-17-methyl-4-(propionyloxy)-7,8-didehydromorphinan-6-yl propionate and addition salts thereof with a pharmaceutically-acceptable acid or base.

48. A compound of claim 31, which is selected from (9α,13α)-1-bromo-3,7-dimethoxy-17-methyl-7,8-didehydromorphinan-4,6-diol and addition salts thereof with a pharmaceutically-acceptable acid or base.

49. A compound of claim 31, which is selected from (9α,13α)-1-bromo-4-hydroxy-3,7-dimethoxy-17-methyl-7,8-didehydromorphinan-6-one oxime and addition salts thereof with a pharmaceutically-acceptable acid or base.

50. A compound of claim 31, which is selected from (9α,13α)-1-bromo-4-hydroxy-3,7-dimethoxy-17-methyl-7,8-didehydromorphinan-6-one N-oxide and addition salts thereof with a pharmaceutically-acceptable acid or base.

51. A compound of claim 31, which is selected from (9α,13α)-1-chloro-4-hydroxy-3,7-dimethoxy-17-methyl-7,8-didehydromorphinan-6-one N-oxide and addition salts thereof with a pharmaceutically-acceptable acid or base.

52. A compound of claim 31, having the configuration shown by formula (I′): 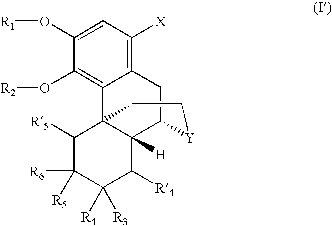
and addition salts thereof with a pharmaceutically-acceptable acid or base.

53. A method for treating a living animal body, including a human, afflicted with a condition selected from deficiencies of memory associated with cerebral aging and neurodegenerative diseases including Alzheimer's disease, Parkinson's disease, Pick's disease, Korsakoff's disease, and frontal lobe and subcortical dementias, comprising the step of administering to the living animal body, including a human, an amount of a compound of claim 31 which is effective for the alleviation of the condition.

54. A pharmaceutical composition useful for treating deficiencies of memory associated with cerebral ageing and neurodegenerative diseases including Alzheimer's disease, Parkinson's disease, Pick's disease, Korsakoff's disease, and frontal lobe and subcortical dementias, comprising as active principal an effective amount of a compound of claim 31 together with one or more pharmaceutically-acceptable excipients or vehicles.

55. A method for treating a living animal body, including a human, afflicted with a condition selected from deficiencies of memory associated with cerebral aging and neurodegenerative diseases including Alzheimer's disease, Parkinson's disease, Pick's disease, Korsakoff's disease, and frontal lobe and subcortical dementias, comprising the step of administering to the living animal body, including a human, an amount of sinomenine and/or a sinomenine compound which is effective for alleviation of the condition. cm 56. The method of claim 55, comprising administering a pharmaceutically effective amount of sinomenine.

57. The method of claim 55, comprising administering a pharmaceutically effective amount of a sinomenine compound.

58. The method of claim 55, wherein the sinomenine compound is selected from those of formula (Ia): 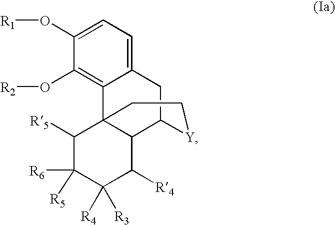
wherein
R₁ represents alkyl,
R₂ represents hydrogen, alkylcarbonyl, haloalkylcarbonyl or arylcarbonyl,
Y represents 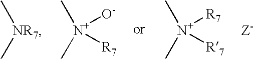
wherein R₇ and R′₇, which may be identical or different, each represent alkyl and Z⁻ represents a halogen anion,
R₃ represents hydroxy or alkoxy,
R₄ and R′₄ each represent hydrogen or together form an additional bond, or R₃ and R₄ together form oxo or ═N—OR₈ (wherein R₈ represents hydrogen or alkyl),
R₆ represents hydroxy, alkylcarbonyloxy (wherein the alkyl moiety may be substituted by hydroxy, alkoxy, carboxy or alkyloxycarbonyl) or alkoxy,
R₅ and R′₅ each represent hydrogen or together form an additional bond, or R₅ and R₆ together form oxo, ═N—OR₉ or ═N—NR₁₀R₁₁ (wherein R₉, R₁₀, and R₁₁, which may be the same or different, each represent hydrogen or alkyl),
it being understood that:
the term “alkyl” denotes an alkyl group having 1 to 6 carbon atoms which may be linear or branched and
the term “alkoxy” denotes an alkyloxy group having 1 to 6 carbon atoms which may be linear or branched,
its enantiomers and diastereoisomers, and addition salts thereof with a pharmaceutically-acceptable acid or base.

59. The method of claim 55, wherein the sinomenine compound is selected from (9α,13α)-4-hydroxy-3,7-dimethoxy-17-methyl-7,8-didehydromorphinan-6-one hydrazone;
(7α,9α,13α)-4-hydroxy-3,7-dimethoxy-17-methylmorphinan-6-one;
(7β,9α,13α)-4-hydroxy-3,7-dimethoxy-17-methylmorphinan-6-one;
(9α,13α)-3,7-dimethoxy-17-methyl-6-oxo-7,8-didehydromorphinan-4-yl propionate;
(9α,13α)-3,4,7-trimethoxy-17-methyl-7,8-didehydromorphinan-6-one;
(9α,13α)-4-hydroxy-3,7-dimethoxy-17-methyl-7,8-didehydromorphinan-6-one oxime;
(9α,13α)-3,7-dimethoxy-17-methyl-7,8-didehydromorphinan-4,6-diol;
(9α,13α)-4-hydroxy-3,7-dimethoxy-17-methyl-7,8-didehydromorphinan-6-one N-oxide;
(9α,13α)-6-amino-3,7-dimethoxy-17-methylmorphinan-4-ol;
4-{[(9α,13α)-4-hydroxy-3,7-dimethoxy-17-methyl-7,8-didehydromorphinan-6-yl]-oxy}-4-oxobutanoic acid;
(9α,13α)-3,7-dimethoxy-17-methyl-4-(propionyloxy)-7,8-didehydromorphinan-6-yl propionate;
(9α,13α)-17-benzyl-4-hydroxy-3,7-dimethoxy-17-methyl-7,8-didehydromorphinan-17-ium-6-one bromide;
(9α,13α)-17-ethyl-3,7-dimethoxy-17-methyl-7,8-didehydromorphinan-17-ium4,6-diol bromide;
(9α,13α)-17-ethyl-4-hydroxy-3,7-dimethoxy-17-methyl-7,8-didehydromorphinan-17-ium-6-one bromide;
(9α,13α)-4-(benzoyloxy)-3,7-dimethoxy-17-methyl-7,8-didehydromorphinan-6-yl benzoate;
(9α,13α)-4-hydroxy-3,7-dimethoxy-17-methyl-7,8-didehydromorphinan-6-yl benzoate; and
(9α,13α)-6-hydroxy-3,7-dimethoxy-17-methyl-7,8-didehydromorphinan-4-yl benzoate.

60. A pharmaceutical composition for use in the treatment of deficiencies of memory associated with cerebral ageing and neurodegenerative diseases including Alzheimer's disease, Parkinson's disease, Pick's disease, Korsakoff's disease, and frontal lobe and subcortical dementias comprising as active principle an effective amount of sinomenine or a sinomenine compound, together with one or more pharmaceutically-acceptable excipients or vehicles.
